(19) United States
(12) Patent Application Publication
(10) Pub. No.: US 2025/0391520 A1
(43) Pub. Date: Dec. 25, 2025

(54) Blockchain-Based System for Secure, Interoperable, and Patient-Centric Electronic Medical Records

(71) Applicant: Chukwuemeka Anderson Obi, Jacksonville, FL (US)

(72) Inventor: Chukwuemeka Anderson Obi, Jacksonville, FL (US)

(21) Appl. No.: 18/748,312

(22) Filed: Jun. 20, 2024

(51) Int. Cl. G16H 10/00; H04L 9/00; H04L 9/06; H04L 9/08

(52) U.S. Cl. CPC G16H 10/00; H04L 9/0643; H04L 9/0861; H04L 9/0891; H04L 9/50

(57) ABSTRACT

Systems and methods herein introduce a blockchain-based system for managing electronic medical records (EMRs) with a focus on enhancing security, interoperability, and patient-centric control. Employing advanced encryption techniques, smart contracts, and a tailored consensus mechanism, the system ensures the integrity, privacy, and efficient exchange of medical data. Patients are empowered with granular control over data access and usage, while interoperability is achieved through standardized data formats and communication protocols. Addressing critical shortcomings of traditional EMR systems, the invention provides a tamper-evident audit trail for transparency and accountability within the healthcare ecosystem.

### BACKGROUND

### Field of the Invention

Embodiments of the present invention pertain to the domain of digital health records management and blockchain technology. It offers a method and system designed to securely, efficiently, and in a patient-centric manner store, retrieve, and manage electronic medical records, leveraging blockchain technology for interoperability.

### Description of the Related Art

Blockchain technology has evolved greatly since the introduction of Bitcoin in 2008, the first decentralized peer-to-peer electronic cash system. Today, innovators in various fields are realizing the benefits of the technology behind Bitcoin. From medicine to finance, many sectors are looking for ways to integrate blockchain into their infrastructures.

With its decentralized and trustless nature, Blockchain technology can lead to new opportunities and benefit businesses through greater transparency, enhanced security, and easier traceability.

Blockchain is far more secure than other record keeping systems because each new transaction is encrypted, transparency and linked to the previous transaction. Blockchain, as the name suggests, is formed by a network of computers coming together to confirm a ‘block’, this block is then added to a ledger, which forms a ‘chain’. Blockchain is formed by a complicated string of mathematical numbers and is impossible to be altered once formed. This immutable and incorruptible nature of blockchain makes it safe from falsified information and hacks. It's decentralized nature also gives it a unique quality of being ‘trustless’-meaning that parties do not need trust to transact safely.

In the realm of digital health records management, blockchain technology presents a paradigm shift. Its decentralized nature ensures that sensitive medical data remains secure and immutable, reducing the risk of tampering or unauthorized access. By distributing records across a network of nodes, blockchain enhances data integrity and transparency, crucial aspects in healthcare where accuracy and privacy are paramount.

The importance of blockchain in digital health records management is underscored by its ability to address several longstanding challenges in the healthcare industry. Data interoperability, often hindered by disparate systems and incompatible formats, can be significantly improved through blockchain's standardized protocols and decentralized architecture. Moreover, blockchain facilitates secure data sharing among healthcare providers, empowering patients with greater control over their medical information.

Facts and figures highlight the growing adoption of blockchain in healthcare. Reports indicate a steady increase in investments and initiatives aimed at integrating blockchain solutions into health systems worldwide. For instance, by 2021, the global blockchain healthcare market was estimated to be worth billions, with forecasts projecting exponential growth in the coming years.

Looking ahead, the future outlook for blockchain technology in digital health records management appears promising. As advancements in blockchain infrastructure and regulatory frameworks continue, more healthcare organizations are expected to embrace blockchain solutions to streamline operations, enhance data security, and improve patient care outcomes. With ongoing research and development initiatives focused on optimizing blockchain's capabilities for healthcare applications, the technology is poised to revolutionize the industry.

Several patents have been filed that merge blockchain technology with health record management, showcasing innovation in this burgeoning field. One such patent, filed by IBM, outlines a method for managing medical data securely using a blockchain network. The system ensures privacy by encrypting patient data and granting access only to authorized users through cryptographic keys. Moreover, the blockchain ledger maintains a transparent and tamper-proof record of medical transactions, enhancing trust and data integrity within the healthcare ecosystem.

Another notable patent, attributed to Microsoft, describes a blockchain-based platform for healthcare data management. The system leverages smart contracts to automate and streamline various processes, such as patient consent management and data sharing agreements. Through the immutable nature of blockchain, the platform ensures the integrity and traceability of health records, facilitating seamless collaboration among healthcare providers while maintaining patient privacy and compliance with regulatory standards.

In addition to industry giants, smaller entities have also contributed to the patent landscape in blockchain and health record management. A startup called Healthereum has patented a blockchain-based incentivization system aimed at improving patient engagement and compliance with healthcare protocols. The platform rewards patients with tokens for completing tasks such as scheduling appointments or adhering to treatment plans, fostering a more proactive approach to healthcare management while leveraging the transparency and security of blockchain technology.

These patents represent just a fraction of the ongoing innovation at the intersection of blockchain and health record management. As more stakeholders recognize the potential of blockchain to address challenges in healthcare data interoperability, security, and patient engagement, the patent landscape is likely to continue evolving with new technologies and applications aimed at transforming the delivery and management of healthcare services. The current invention proposes a system which is the apparatus and method tailored for integrating Electronic Medical Records (EMRs) onto a blockchain network, prioritizing heightened security, privacy, interoperability, and patient-centric control. Utilizing smart contracts, the system streamlines access control, manages data sharing agreements, and ensures adherence to healthcare regulations. Advanced encryption and anonymization techniques bolster data privacy, fortifying patient confidentiality within the network. Furthermore, a bespoke consensus mechanism is implemented to optimize transaction efficiency, bolster security, and uphold regulatory compliance within the intricate landscape of healthcare data management.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

It is therefore the purpose of the invention to alleviate at least to some extent one or more of the aforementioned problems of the prior art and/or to provide the relevant public with a suitable alternative thereto having relative advantages.

The primary object of the invention is related to the provision of a to enhance data security by leveraging blockchain technology, ensuring that electronic medical records (EMRs) are tamper-proof and resistant to unauthorized access.

Another desirable object of the present invention is to provide a novel and improved form of a system that prioritize patient-centric control, allowing individuals greater autonomy over their medical data within the blockchain network.

It is also the objective of the invention to provide improved interoperability among healthcare providers by establishing standardized protocols for sharing EMRs across disparate systems.

It is moreover the objective of the invention to enhance compliance with healthcare regulations by embedding regulatory requirements within the blockchain network's operations.

It is moreover the objective of the invention to optimize transaction efficiency within the healthcare context, facilitating swift and secure exchanges of EMRs among authorized parties.

It is further the objective of the invention to bolster patient privacy through advanced encryption and anonymization techniques, safeguarding sensitive medical information from unauthorized disclosure.

It is also the objective of the invention to foster trust and transparency within the healthcare ecosystem by maintaining a transparent and immutable record of medical transactions on the blockchain ledger.

It is further the objective of the invention to reduce administrative overhead and costs associated with traditional health record management systems by implementing a more efficient and decentralized approach.

It is moreover the objective of the invention to provide a system that paves the way for future innovations in healthcare data management, offering a scalable and adaptable solution to the evolving needs of the industry.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The current invention in its preferred embodiment introduces a pioneering system and method for integrating Electronic Medical Records (EMRs) onto a blockchain network, aimed at substantially enhancing security, privacy, interoperability, and patient-centric control within the healthcare domain. At the core of this innovative approach is the utilization of smart contracts, which automate critical functions including access control, data sharing agreements, and compliance with healthcare regulations such as HIPAA and GDPR. By harnessing advanced encryption and anonymization techniques, the system ensures the utmost protection of sensitive medical data, safeguarding patient privacy while simultaneously fortifying data integrity and security.

As per its further embodiments, in terms of system architecture, the invention establishes a decentralized blockchain network where EMRs are securely stored, accessible solely by authorized users including healthcare providers, patients, and approved third parties. This distributed setup not only enhances data redundancy and availability but also fosters distributed trust, mitigating the risk of single points of failure or malicious manipulation. Furthermore, the architecture is designed to support a granular level of patient control over data access and usage, empowering individuals with the autonomy to manage their medical information as they see fit.

As per its additional embodiments, the method of storing EMRs on the blockchain involves several key steps to ensure data privacy, integrity, and controlled access. Before storage, EMRs undergo encryption using advanced cryptographic techniques, rendering them indecipherable to unauthorized entities. Each EMR is assigned a unique hash, stored on the blockchain, enabling verification of data integrity without exposing sensitive information. Smart contracts play a pivotal role in managing EMR access, enforcing predefined rules and requiring explicit patient consent before data sharing occurs. Additionally, mechanisms for tracking EMR data lineage and modifications are implemented, facilitating auditability and supporting research endeavors.

As per its further embodiments, interoperability is a fundamental aspect addressed by the invention, achieved through the establishment of standardized data formats and communication protocols compatible with diverse healthcare systems and providers. By maintaining a decentralized directory of EMR schemas and mappings, the blockchain network facilitates seamless data exchange and integration, overcoming the interoperability challenges inherent in traditional EMR systems.

As per its additional embodiments, the consensus mechanism employed within the blockchain network is custom-designed to suit the unique requirements of healthcare data transactions. Drawing upon principles such as Practical Byzantine Fault Tolerance (PBFT), the mechanism strikes a delicate balance between transaction efficiency, security, and compliance obligations, potentially incorporating incentive structures to encourage active participation within the network.

As per its further embodiments, privacy and security measures are paramount within the invention, with advanced encryption techniques safeguarding data both at rest and in transit. Anonymization techniques, such as differential privacy or homomorphic encryption, are explored to enable research and analytics without compromising patient privacy. Additionally, tamper-evident audit trails are established to provide transparency regarding data usage, reinforcing accountability and trust within the healthcare ecosystem.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1. A method for securely storing electronic medical records (EMRs) on a blockchain network, comprising:
The method as per claim 1, wherein encrypting EMR data using a provably secure symmetric encryption algorithm with a minimum key length of 256 bits, with a commitment to evolve encryption techniques in line with emerging cryptography standards;
The method as per claim 1, wherein generating a SHA-256 hash of the encrypted EMR data for integrity verification;
The method as per claim 1, wherein storing the encrypted EMR data and the associated hash on the blockchain network;
The method as per claim 1, wherein employing a hardware security module (HSM) meeting FIPS 140-2 Level 3 or higher, defining the following key management procedures:
The method as per claim 1, wherein secure key generation within the HSM including auditable processes for key derivation or importation.
The method as per claim 1, wherein periodic key rotation based on a configurable schedule and event-triggered mechanisms (e.g., following potential compromise).
The method as per claim 1, wherein key revocation mechanism that securely renders revoked keys unusable and generates detailed logs for audit purposes.

2. A system for decentralized EMR management on a blockchain network, comprising:
The method as per claim 2, wherein nodes operated by healthcare providers, patients, and authorized third parties, with a robust verification process for onboarding new participants;
The method as per claim 2, wherein smart contracts defining includes a granular, role-based access control system customizable to the specific workflow and regulatory requirements of a healthcare setting;
The method s per claim 2, wherein mandatory data sharing agreements governed by patient consent with granular revocation rights, where revocation requests are themselves immutably recorded on the blockchain; and,
The method as per claim 2, wherein an auditable logging system tracking all data access, modifications, and sharing, aligned with HIPAA and GDPR regulations, with logs immutably stored on the blockchain and linked to corresponding regulatory compliance verification events.

3. A method for interoperable EMR exchange within the blockchain network, comprising:
The method as per claim 3, wherein utilizing the FHIR standard for representing and structuring EMR data as a foundational framework;
The method as per claim 3, wherein the system provides mechanisms to register, translate, and utilize alternative healthcare data representation standards when necessitated by cross-system compatibility;
The method as per claim 3, wherein secure communication and exchange of EMRs between nodes using a RESTful API, including TLS 1.2 or higher for transport layer security;
The method as per claim 3, wherein a blockchain-based directory for dynamic registration of data schemas and their mappings, enabling seamless interoperability across diverse healthcare systems; and,
The method as per claim 3, wherein a directory is designed to reconcile different standards by including robust, auditable translation mechanisms.

4. A tailored consensus mechanism for healthcare transactions on the blockchain network, comprising:
The method as per claim 4, wherein a consensus mechanism derived from the Practical Byzantine Fault Tolerance (PBFT) algorithm, with the following optimizations specifically designed for permission healthcare settings;
The method as per claim 4, wherein a reduction in message complexity minimizes communication overhead;
The method as per claim 4, wherein incorporation of on-chain, automated regulatory compliance checks tailored to relevant healthcare regulations (e.g., HIPAA) as an integral part of the consensus process; and,
The method as per claim 4, wherein an incentive structure using a native token that rewards nodes for both validation and active participation in ensuring regulatory compliance, encouraging network integrity and adherence to standards.

5. A robust privacy protection for EMR data on the blockchain network, comprising:
The method as per claim 5, wherein by incorporating differential privacy techniques to enable statistical analysis of EMR datasets while preserving the privacy of individual patient information, ensuring compliance with privacy regulations and protecting sensitive data; and,
The method as per claim 5, wherein by implementing homomorphic encryption to enable computations on encrypted EMR data without decryption, preserving data privacy and confidentiality while allowing for secure data processing and analysis.

6. A method for tamper-evident audibility within the blockchain network, comprising:
The method as per claim 6, wherein by generating immutable and timestamped log entries for each access, modification, or sharing event of EMR data, providing a comprehensive audit trail for traceability and accountability; and,
The method as per claim 6, wherein by cryptographically signing log entries with node-specific keys to verify the authenticity and origin of each entry, enhancing the security and trustworthiness of the audit trail.

7. A patient-centric control mechanism over EMR data in the blockchain network, comprising:
The method as per claim 7, wherein by providing a user-friendly interface for patients to control access permissions to their EMR data, empowering patients to manage their data with autonomy and transparency; and,
The method as per claim 7. wherein by granting patients granular control over access to specific segments of their EMR data, including the ability to define time-limited access periods and view a transparent log of data usage, promoting transparency and enhancing patient empowerment.
